(19) United States
(12) Patent Application Publication
(10) Pub. No.: US 2022/0202347 A1
(43) Pub. Date: Jun. 30, 2022

(54) SYSTEMS AND METHODS FOR NEUROLOGICAL DISORDER DETECTION

(71) Applicants: Imienitie Arhuidese, San Francisco, CA (US); Onon Arhuidese, Chapel Hill, NC (US)

(72) Inventors: Imienitie Arhuidese, San Francisco, CA (US); Onon Arhuidese, Chapel Hill, NC (US)

(21) Appl. No.: 17/566,835

(22) Filed: Dec. 31, 2021

Related U.S. Application Data
(60) Provisional application No. 63/132,614, filed on Dec. 31, 2020.

(51) Int. Cl. A61B 5/374; A61B 5/291; A61B 5/00

(52) U.S. Cl. CPC A61B 5/374; A61B 5/291; A61B 5/7257; A61B 5/7246; A61B 5/7264

(57) ABSTRACT

A system for detecting neurological disorders based on an electroencephalogram (EEG) signal is disclosed. The system includes at least one processor and at least one memory storing instructions. The instructions, when executed by the at least one processor, cause the system to access the EEG signal, process the EEG signal to filter for a brainwave frequency band, detect, via an EEG analytic engine, a brainwave pattern from the processed EEG signal based on the filtered brainwave frequency band, and generate, via a synchronization pattern visual generator, a two-dimensional network pattern image of the detected brainwave pattern.

### CROSS-REFERENCE TO RELATED APPLICATIONS

This application claims the benefit of, and priority to, U.S. Provisional Patent Application No. 63/132,614, filed on Dec. 31, 2020, titled: Systems And Method For Neurological Disorder Detection.

### TECHNICAL FIELD

The present disclosure relates to systems and methods for detecting neurological disorders, and specifically, for determining multiscale synchronization or brainwave patterns within disrupted neural networks of neural connectivity-based diseases.

### BACKGROUND

Current clinical methods for determining the onset and diagnoses of neural connectivity-based diseases, such as Autism Spectrum Disorder (ASD), schizophrenia, bipolar disorder, attention deficit hyperactivity disorder, severe depression, etc., are often based on anecdotal, historical, and clinical examinations. The current clinical diagnostic methods of neural connectivity-based diseases do not accurately pinpoint or identify the actual abnormal neurological patterns of brainwaves within the brain that may be associated with a neural connectivity-based disease. It is still unknown how early in development (e.g., as a baby, adolescent, or adult) synchronization or desynchronization of neurons that may cause such neural connectivity-based diseases emerges and what their impact is on behavioral symptoms. The diagnosis of such neural connectivity-based diseases is therefore often difficult to predict or detect without sufficient anecdotal, historical, or clinical examinations and may often be misdiagnosed or not diagnosed as early as possible for sufficient and/or the most effective clinical intervention.

Some of the existing methods to identify ASD and similar neural connectivity-based diseases include the deployment of Functional Magnetic Resonance Imaging (FMRI) systems to measure brain activity. FMRI is used to pick up locations of abnormal neural activity by detecting changes in the brain based on blood flow. Inferences are made based on the amount of oxygenated blood flow changes (also referred to as Blood-Oxygenated Level Dependent response or BOLD). FMRI is bulky, inflexible, and inadequate to measure neural events (i.e., brain activity) directly based on the flow of electrical current or due to the flow of electrical charges separated across the outer membrane of the millions of neurons of the brain. What is needed is a system and methods thereof for determining the onset and diagnosis of neural connectivity-based diseases based on electrical brainwave activity.

### SUMMARY

In accordance with aspects of the disclosure, a system for detecting neurological disorders based on an electroencephalogram (EEG) signal. The system includes at least one processor and at least one memory storing instructions thereon. The instructions, when executed by the at least one processor, cause the system to: access the EEG signal; process the EEG signal to filter for a brainwave frequency band; detect, via an EEG analytic engine, a brainwave pattern from the processed EEG signal based on the filtered brainwave frequency band; and generate, via a synchronization pattern visual generator, a two-dimensional network pattern image of the detected brainwave pattern. Other aspects, features, and advantages will be apparent from the description, the drawings, and the claims that follow.

The system may include one or more of the following features. The at least one memory may store further instructions which, when executed by the at least one processor, may cause the system to generate a three-dimensional spatial graphic with the detected brainwave pattern superimposed thereon. The three-dimensional spatial graphic may be a three-dimensional model of a brain having an amount of nodes equal to an amount of EEG sensors used to detect the EEG signal. The EEG signal may be accessed in real-time from an EEG device. The instructions further may include at least one of the following steps: generating a plurality of two-dimensional network pattern images of the detected brainwave pattern as a function of time; or generating a plurality of three-dimensional spatial graphics with the detected brainwave pattern superimposed thereon as a function of time. The instructions to process the EEG signal may include curating and/or transforming time series data of the EEG signal, extracting at least one non-linear invariant measure from the EEG signal, generating at least one recurrence plot of the EEG signal, and/or generating at least one correlation matrix relating the EEG signal to a plurality of sensors of an EEG device. The at least one memory may store further instructions which, when executed by the at least one processor, cause the system to display the two-dimensional spatial graphic. The instruction to process the EEG signal may include applying a Fourier transform. The two-dimensional network pattern image of the detected brainwave pattern may include a map of a plurality of sensors, each sensor of the plurality of sensors configured to provide a portion of the EEG signal. The EEG analytic engine may detect the brainwave pattern based on the correlation matrix. The EEG analytic engine may be a classical machine learning classifier, a convolutional neural network, a deep learning network, an associative, a non-associative, or a clustering machine learning system.

This disclosure also provides a computer-implemented method for detecting neurological disorders based on an electroencephalogram (EEG) signal. The computer-implemented method may include accessing an EEG signal; processing the EEG signal to filter for a brainwave frequency band; detecting, via an EEG analytic engine, a brainwave pattern from the processed EEG signal based on the filtered brainwave frequency band; and generating, via a synchronization pattern visual generator, a two-dimensional network pattern image of the detected brainwave pattern.

Implementations of the method may include one or more of the following features. The computer-implemented method may include generating a three-dimensional spatial graphic with the detected brainwave pattern superimposed thereon. The three-dimensional spatial graphic may be a three-dimensional model of a brain having a plurality of nodes that may correspond to a plurality of sensors of an EEG device. The computer-implemented method may include at least one of the following steps: extracting a non-linear invariant measure from the EEG signal; generating a recurrence plot of the EEG signal; or generating a correlation matrix relating the EEG signal to a plurality of sensors of an EEG device. Processing the EEG signal may include applying a Fourier transform. The computer-implemented method may include designating, via the EEG analytic engine, a spatial correlation between a plurality of neural circuits associated with a plurality of sensors of an EEG device from which the EEG signal was sensed. The EEG signal may be accessed in real-time from an EEG device. Generating the two-dimensional network pattern image may include generating a plurality of two-dimensional network pattern images as a function of time.

### BRIEF DESCRIPTION OF THE DRAWINGS

The accompanying drawings, which are incorporated in and constitute a part of this specification, illustrate aspects of the disclosure and, together with a general description of the disclosure given above and the detailed description given below, serve to explain the principles of this disclosure, wherein:

FIG. 1 is a block diagram of a neurological disorder visualizer and detector system, in accordance with the present disclosure;

FIG. 2 is a block diagram of a computing device of the neurological disorder visualizer and detector system of FIG. 1;

FIGS. 3A-C are diagrams of correlation coefficients for EEG sensor locations for theta, alpha, and gamma brainwave frequency bands for average and Autism Spectrum Disorder (ASD) groups;

FIGS. 4A-C are diagrams of two-dimensional synchronization of subregions of a brain based on theta, alpha, and gamma brainwave frequency bands;

FIGS. 5A-C are diagrams of three-dimensional spatial graphics of a model of a brain with an alpha brainwave pattern superimposed thereon; and

FIG. 6 is a flowchart illustrating an exemplary method according to the present disclosure.

### DETAILED DESCRIPTION

The present disclosure relates to systems and methods for detecting neurological disorders and provides a neurological disorder visualizer and detector.

The term “computer,” “computing device,” “mobile device,” “server” may refer to a computer including a processor and a memory, which include processor-executable instructions. When the processor executes the processor-executable instructions, the computer performs any features or functions to provide functionalities of this disclosure. The computer may be a local or remote server.

The term “application,” “module,” “unit,” and “software” may include a computer program designed to perform particular or specific functions, tasks, or activities for the benefit of a user. Application may refer to, for example, software running locally or remotely, as a standalone program or in a web browser, or other software which would be understood by one skilled in the art to be an application. An application may run on a controller or on a user device, including, for example, on a mobile device, an internet-of-thing (IoT) device, or a server system.

This disclosure helps to address the need to determine a critical window at which neural connectivity-based diseases, such as Autism Spectrum Disorder (ASD), manifest during periods of a person's development (e.g., in utero, as a baby, adolescent or adult). It also provides advantageous images and/or graphics of brainwave patterns not previously attainable. The disclosure provides a neurological disorder visualizer and detector system (NeDVDS) and methods thereof that provide visuals into the neural paths that may fire synchronously or asynchronously and detects and associates brainwave patterns with a particular neural connectivity based disease.

Brainwaves are produced by the electrical pulses of neurons of a brain communicating with each other. Brainwaves are commonly described as the oscillating electrical voltages in the brain delineated across various frequency bands. The various brainwaves (delta, theta, alpha, beta, and gamma) may exhibit various patterns and are generally associated with various brain states (such as concentration, sleep, activity, awake, relaxed, passive attention, etc.), and emotional states. The various brainwaves may dominate at different times or for different activities. For those with neural connectivity-based diseases or disorders, the brainwaves that are dominant or passive for a particular activity or state may be different than those without the neural connectivity-based disease or disorder. Theta brainwaves involve signals transmitted in several cognitive functions including memory and cognitive control. Alpha brainwaves are associated with functions related to wakeful and resting states. Gamma brainwaves are associated with large scale brain network activity and cognitive phenomenon such as working memory, attention, and perception grouping. Thus, if a brainwave pattern can be associated with a neural connectivity-based disease the detection of that pattern may be used by a clinician to diagnose a patient with that disease. However, the neurons of a brain make billions of connections and make detection of brainwave patterns that may be associated with a particular neural connectivity-based disease difficult. The neurological disorder visualizer and detector system of this disclosure enables the determination of these associations of brainwaves to neural connectivity-based diseases to be easily made.

Additionally, researchers, scientists, and clinicians have previously hypothesized that a brain has a default mode network comprised of certain brainwaves that are active or not active in some combination emanating from some combination of various areas of the brain, but have been unable to identify and illustrate a default mode network. The neurological disorder visualizer and detector system of this disclosure is able to identify and illustrate default mode networks of a brain and other brainwave patterns, such as those associated with particular activities, in addition to detecting and illustrating neural connectivity-based disorders via brainwave patterns.

In accordance with this disclosure, delta brainwaves are those between about 0.5 hz and about 4 hz; theta brainwaves are those between about 4 hz and about 7 hz; alpha brainwaves are those between about 8 hz and about 16 hz; beta brainwaves are those between about 16 hz and about 32 hz; and gamma brainwaves are those between about 32 hz and about 64 hz. The frequency range of each delta, theta, alpha, beta, and gamma brainwaves may be defined differently as known by those of ordinary skill in the art without departing from the scope of this disclosure. For example, those of ordinary skill in the art may also define theta waves as those between about 3 and about 8 hz.

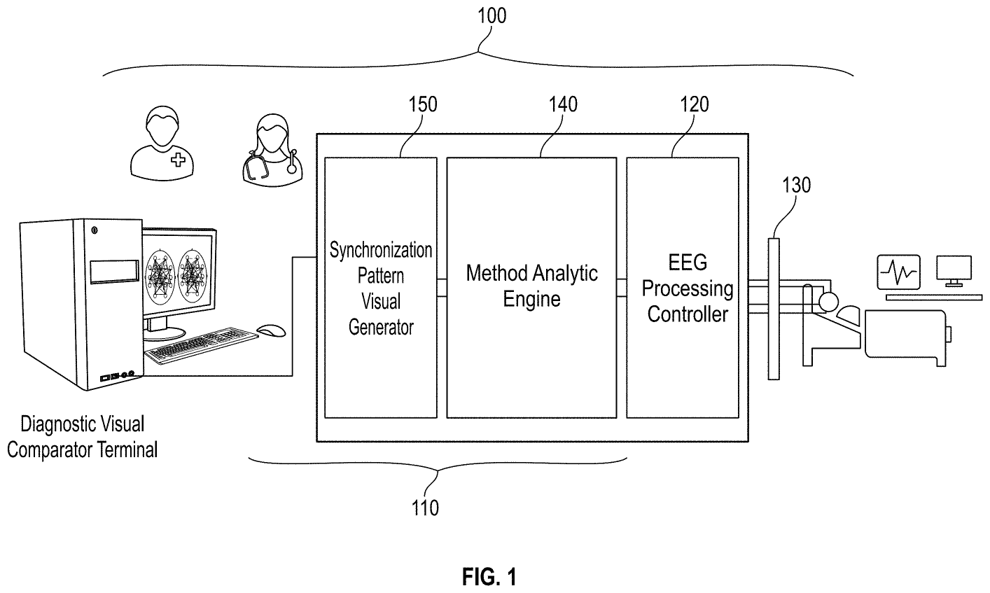

Referring now to FIG. 1, a neurological disorder visualizer and detector system (NeDVDS) 100 includes a server 110 and an electroencephalography (EEG) processing controller 120 configured to receive and/or process electrical signals (EEG signals) detected by an EEG device 130. The server 110 includes an EEG analytic engine 140 and a synchronization pattern visual generator 150.

The EEG device 130 may include one or more sensors (e.g., electrodes) configured to detect the electrical activity of a brain of a person. The EEG device 130 may be any suitable device for detecting electrical activity of a brain known by those of ordinary skill in the art. The NeDVDS 100 capitalizes on the multidimensional characteristics of the electrical signals detected by the EEG device 130. The multidimensional characteristics of the electrical signals, or EEG data, include time, space, frequency, power, and phase.

The NeDVDS 100 is configured to identify neurologic and psychiatric disorders based on electrical brainwave data (or an EEG signal). The electrical brainwave data is captured via the EEG device 130. The NeDVDS 100 analyzes the brainwave data or a signal from an EEG device 130 and extracts a brainwave pattern or brainwave synchronized patterns with signal processing methods to generate brainwave activity visuals near real-time. The NeDVDS 100 may predict the abnormal connectivity issues associated with neural connectivity-based diseases by comparing brainwave activity visuals of a patient with those associated with a known neural connectivity-based disease.

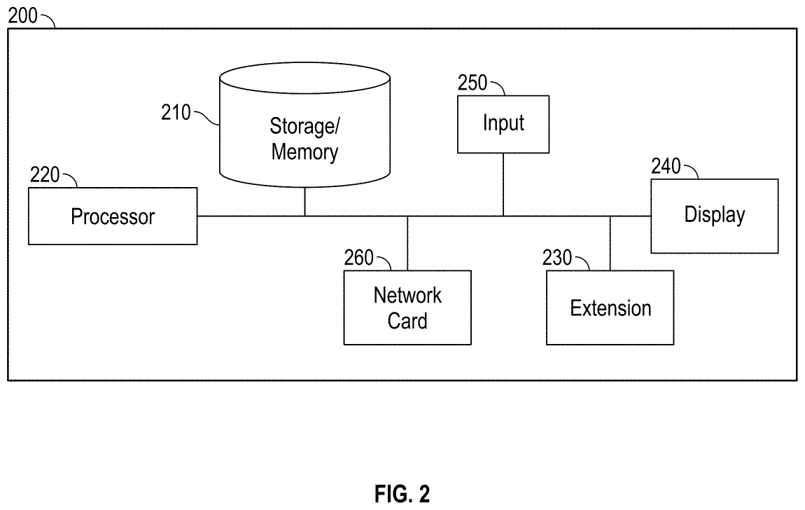

The EEG processing controller 120 is configured to curate and transform time-series EEG data. The EEG data may be accessed by the EEG processing controller 120 stored on a memory device (e.g., 210, FIG. 2) or from the EEG device 130 in real-time. The EEG processing controller 120 is further configured to extract non-linear invariant measures, generate recurrence plots, and/or set up a sensor correlation matrix of signals from sensors, located at multiple regions of a person's brain, of the EEG device 130 that sensed the EEG signal. The EEG processing controller 120 is configured to apply signal processing to the EEG data to translate the low power signals from the EEG device 130. The signal processing applied may include applying Fourier transforms to the signal obtained from the EEG device 130. The EEG processing controller 120 also applies signal filtering at an associated brainwave frequency band. The EEG processing controller 120 may apply a signal filter to isolate delta, theta, alpha, beta, or any combination thereof gamma brainwaves.

The EEG analytic engine 140 receives the processed EEG signal from the EEG processing controller 120. The EEG analytic engine 140 utilizes artificial intelligence or machine learning, including associative (supervised learning), non-associative (unsupervised learning), reinforcement learning, clustering, support vector, decision tree, and/or other machine learning techniques to detect brainwave patterns or synchronization in the processed EEG signal. The EEG analytic engine 140 may be a deep learning neural network, convolutional neural networks (CNN), or other types of artificial intelligence neural networks known by those of ordinary skill in the art. The EEG analytic engine 140 uses machine learning to characterize the EEG data and may designate spatial correlations between neural circuits in the regions of the brain the sensors of the EEG were placed.

The EEG analytic engine 140 associates a detected brainwave pattern with a neural connectivity-based disease. The EEG analytic engine 140 may detect a brainwave pattern across several brainwave frequency bands. Common brainwave patterns across frequency bands may be associated with a neural connectivity-based disease, neurological activity state, and/or a default mode network state. The EEG analytic engine 140 may be a deep learning neural network, convolutional neural networks (CNN), or other types of artificial intelligence neural networks known by those of ordinary skill in the art.

The EEG analytic engine 140 feeds the detected brainwave patterns to the synchronization pattern visual generator 150. The synchronization pattern visual generator 150 is configured to generate a two-dimensional map of the sensors of the EEG device 130 and apply the brainwave pattern to the map. The map may be displayed on a user device, such as a smartphone, personal computer, laptop, tablet, or the like. Clinicians can review the maps to diagnose a patient and/or utilize the map to develop a common map for a particular neural connectivity-based disorder.

The EEG analytic engine 140 may detect the brainwave patterns in real-time and the synchronization pattern visual generator 150 may produce a stream of maps of the brainwave patterns in real-time. The maps may include other brainwave connections or edges (similar to that used to illustrate machine learning methods) occurring at the same time as the determined brainwave pattern.

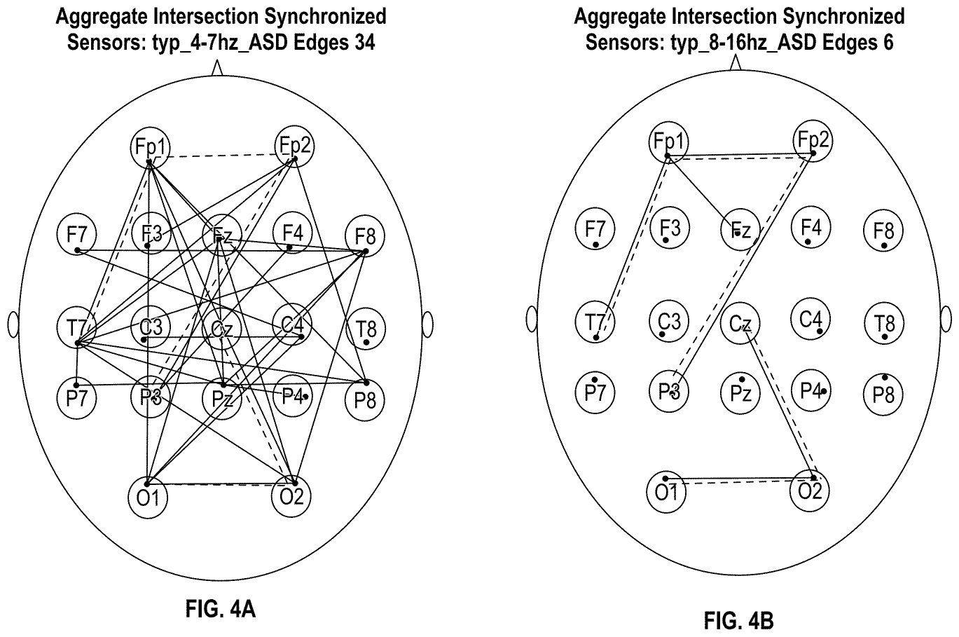

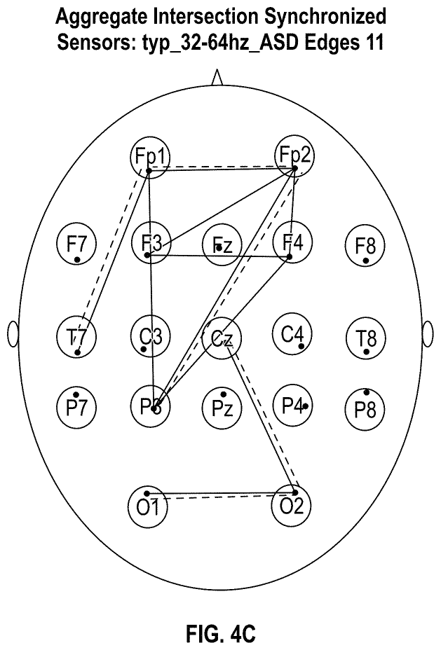

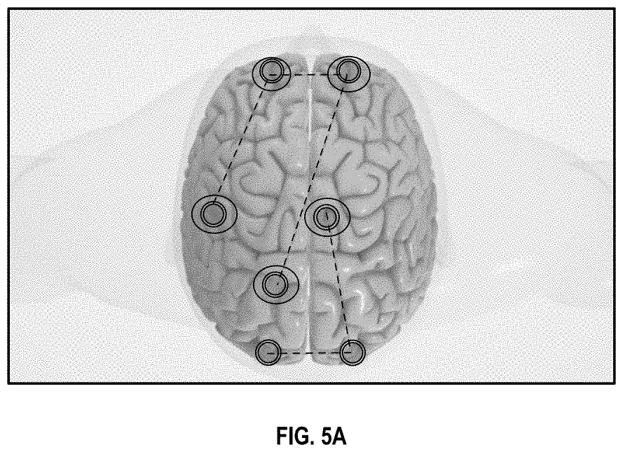

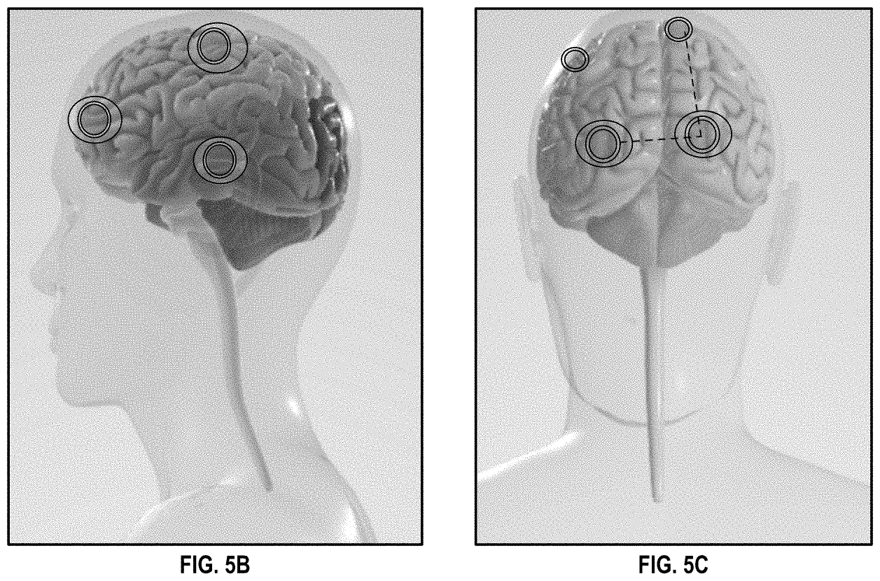

The synchronization pattern visual generator 150 generates the patterns based on an EEG map, such as those shown in FIGS. 4A-C. FIGS. 4A-C illustrate examples of brainwave patterns applied to an EEG map by the synchronization pattern visual generator 150 across theta, alpha, and gamma brainwaves. In the maps, ‘f’ stands for frontal lobe, ‘p’ stands for parietal lobe, T stands for temporal lobe, ‘c’ stands for central lobe, and ‘o’ stands for occipital lobe. The number indicates if the electrode is on the left or right side of the brain, with odd numbers indicating the left side of the brain, even numbers indicating the right side the brain, and ‘z’ indicating the middle of the brain. The generated two-dimensional EEG maps may be compared to standard certified normal patterns such as a hypothetical default mode network and may offer improved clinical data upon which a clinician may base a diagnosis. In FIGS. 4A-C, a common brainwave pattern for an average person (TYP) and a person diagnosed with autism spectrum disorder (ASD) is shown in broken lines and suggests a default mode network. The EEG analytic engine 140 determined this pattern as described above based on a set of EEG signals collected from a sample of TYP and ASD people. The EEG analytic engine 140 suggests that a default mode network between the brain regions T7, FP1, FP2, and P3 and between CZ, O2, and O1 exists for the TYP and ASD person. FIGS. 5A-C illustrate the brainwave pattern identified in the two-dimensional image map superimposed on a three-dimensional model of a brain, where FIG. 5A shows the top of the brain, FIG. 5B shows the left side of the brain, and FIG. 5C is a rear view of the brain. The synchronization pattern visual generator 150 is configured to generate a three-dimensional model of the brain with the brainwave patterns superimposed thereon, such as, but not limited to, those shown in FIGS. 5A-C.

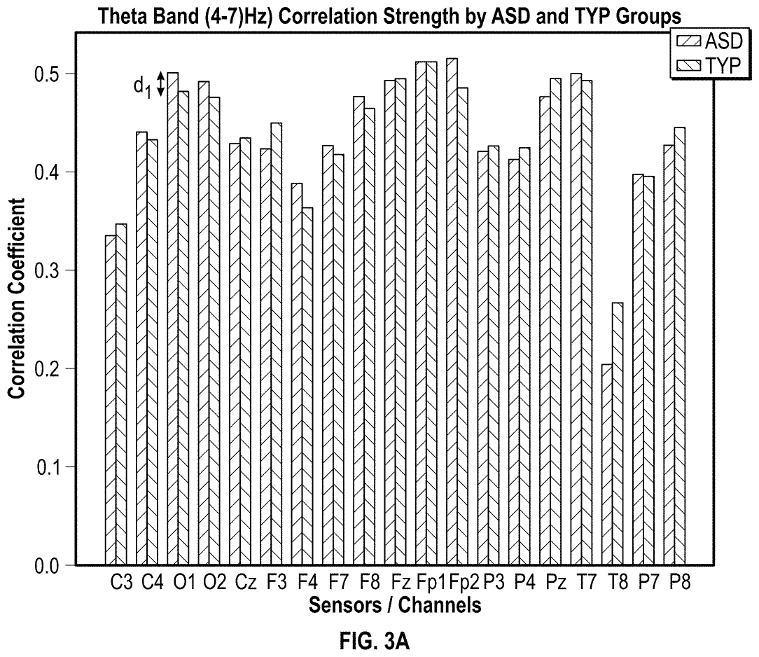

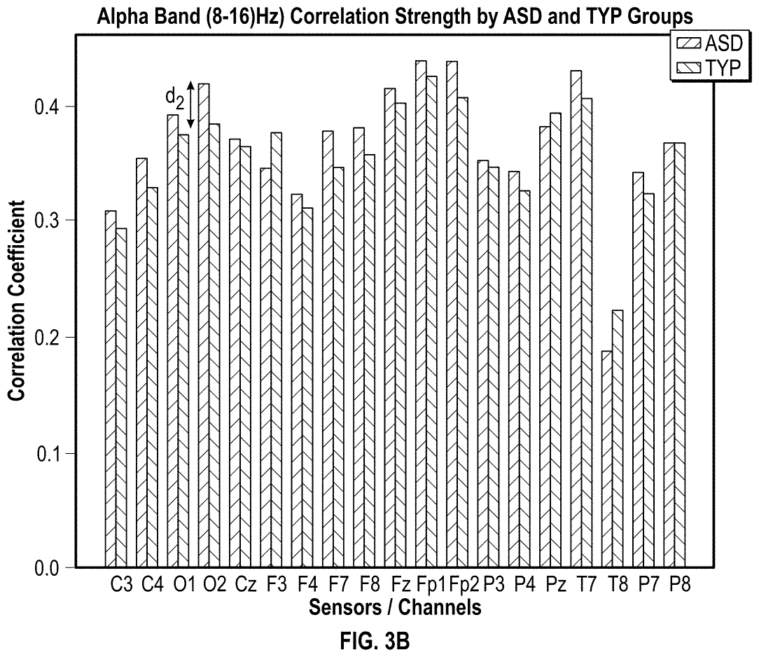

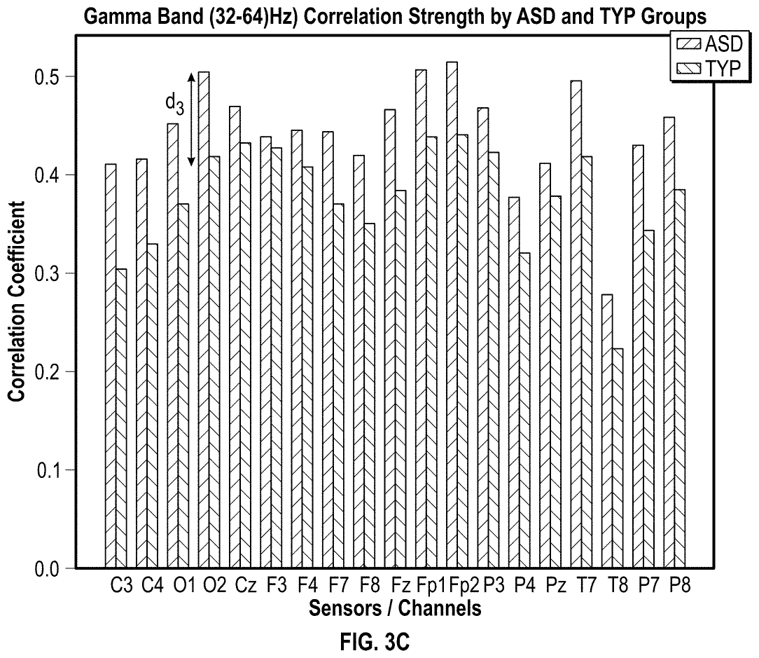

With reference to FIG. 3A-C, the EEG analytic engine 140 may also determine a correlation strength coefficient (y-axis) for each sensor of the EEG device 130 (x-axis). FIG. 3A illustrates a correlation coefficient for theta brainwaves, FIG. 3B illustrates a correlation coefficient for alpha brainwaves, and FIG. 3C illustrates a correlation coefficient for gamma brainwaves. Each graph compares the correlation coefficient for a TYP and ASD person. The difference in strength between the correlation coefficients of a TYP and ASD person may indicate a disorder or departure from the normal electrical activity in the brain at that region. As seen in FIGS. 3A-C, for sensor location O1 (the left occipital lobe), those with ASD may show a greater level of activity in the brain at that location across the theta, alpha, and gamma brainwave bands. Further, as the frequency increased (from theta to gamma brainwave bands), the departure from the normal of a person with an ASD increased, which may be a marker for autism spectrum disorder. The correlation coefficient provides a check on the two-dimensional image map illustrating the brainwave pattern.

The NeDVDS 100 is thus able to show a decline in a patient's clinical condition through detection of brainwave synchronization patterns as they may change over time. For example, it is known that ASD often presents itself after one year of age. The NeDVDS 100 may be used to identify brainwave patterns over time and identify those that signal that ADS may begin to present itself enabling a clinician to intervene and treat the ASD as early as possible (via therapy or other known interventions). The NeDVDS 100 identifies and characterizes brainwave synchronization or patterns within disrupted circuits, such as interhemispheric synchronization, so that it becomes possible to distinguish between healthy & unhealthy brainwave synchronization or patterns, monitor changes over time, and intervene after early detection of the associated brainwave synchronization or patterns.

The NeDVDS 100 is able to detect and associate brainwave patterns or synchronization with neural connectivity-based diseases by testing and creating visualizations of brainwave patterns or synchronization via associative, non-associative, clustered machine learning techniques. The NeDVDS 100, for example, may be used to detect and visualize the brainwave patterns or synchronization of athletes pre- and post-head injury and associate the brainwave patterns with a neural connectivity-based disease (e.g., Chronic Traumatic Encephalopathies or CTE). In another example, the NeDVDS 100 may be used to detect the onset of neural connectivity-based diseases such as Parkinson's disease, tumor infections, genetic brain disorders, mental retardation, cerebral palsy, or hypoxic seizures, and/or used to detect the onset of associated brainwave patterns of other neural connectivity-based disorders.

The NeDVDS 100 may answer questions about brain structure and functional activity in complement with accurate spatial measurements from PET, SPECT, MRI, or CT scans, and/or other similar medical scans. In cases where spatial accuracy of brain components alone is not helpful, the NeDVDS 100 may provide information to differentiate the functional activities of different brain regions at scale. PET, SPECT, MRI or CT scans are dependent on three-dimensional space and provide information on the spatial positioning of brain components but not the functional activity of the different parts of the brain. The NeDVDS 100 complements the spatial accuracy measurements of PET, SPECT, MRI or CT scans by providing a map of functional brain activity of the different areas of the brain by illustrating brainwave patterns, synchronization/desynchronization and connections, since spatial accuracy is not required by the NeDVDS 100 to determine the brainwave patterns. Thus, clinicians can complement the MRI scans with the two-dimensional images generated by the NeDVDS 100.

In aspects, the method provides correlation coefficients that validate the observed associations. The method may use associative, non-associative, and clustering machine learning algorithms. The method provides rendered three-dimensional graphical visualization of brain subregion synchronization patterns.

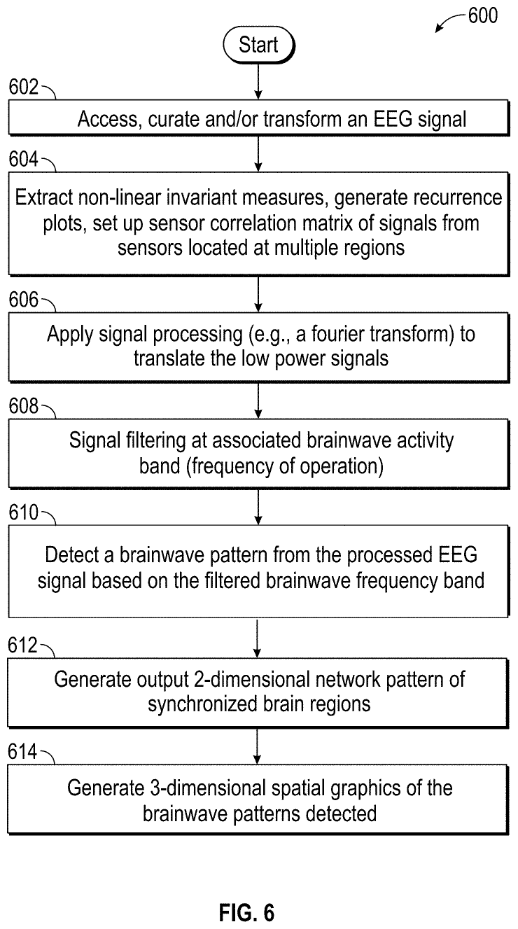

Referring to FIG. 6, there is shown a flow chart of an exemplary computer-implemented method 600 for neurological disorder detection in accordance with aspects of the present disclosure. In accordance with aspects of the present disclosure, the server 110 operates to determine multiscale synchronization within disrupted neural networks of neural connectivity-based diseases (e.g., ASD). Although the operations of the method 600 of FIG. 6 are shown in a particular order, the operations need not all be performed in the specified order, and certain operations can be performed in another order. For simplicity, method 600 will be described below with the server 110 performing the operations. However, in various aspects, the operations of FIG. 6 may be performed in part by the server 110 of FIG. 1 and in part by any other another suitable computing device. These variations are contemplated to be within the scope of the present disclosure.

The method capitalizes on the multidimensional characteristics of EEG signals. The multidimensional characteristics of the EEG data include time, space, frequency, power, and phase. The method includes automatically generating two-dimensional images of neural synchronized subregions compared to standard certified normal patterns such as a hypothetical default mode network. In aspects, the method provides correlation coefficients that validate the observed associations. The method may use associative, non-associative, and clustering machine learning algorithms. The method provides rendered three-dimensional graphical visualization of brain subregion synchronization patterns.

Initially, at step 602, the operation accesses, curates and/or transforms time series EEG signal data obtained from an EEG device connected via cables to a patient's head. Next, at step 604, the operation extracts non-linear invariant measures from the EEG signal, generates recurrence plots, and/or sets up a sensor correlation matrix of signals from sensors located at multiple regions of a brain. At step 606, the operation may apply signal processing such as Fourier transforms to translate the low power signals. At step 608, the operation may apply signal filtering at an associated brain activity band (frequency of operation). At step 610, the operation may feed the resulting EEG signal into an EEG analytic engine 140 to detect a brainwave pattern from the processed EEG signal based on the filtered brainwave frequency band. At step 610, the operation may include designating the spatial correlation between neural circuits in the sensor regions and comparing the detected brainwave pattern with standardized neural connectivity-based disorder patterns. At step 612, the operation may generate and/or output two-dimensional network patterns of synchronized brain regions. At step 614, the operation may generate network circuits in three-dimensional spatial graphics as a function of time.

FIG. 2 is a block diagram for a computing device 200, which may be the server 110 of FIG. 1, in accordance with aspects of the disclosure. The computing device 200 may be connected to one or more external devices and control the external devices. The computing device 200 may include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and embedded computers. Those of skill in the art will recognize that many smartphones are suitable for use in the system described herein. Suitable tablet computers include those with booklet, slate, and convertible configurations, known to those of skill in the art.

In aspects, the computing device 200 includes an operating system configured to perform executable instructions. The operating system is, for example, software, including programs and data, which manage the device's hardware and provide services for the execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In aspects, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In aspects, the computing device 200 includes a storage 210. The storage 210 is one or more physical apparatus used to store data or programs on a temporary or permanent basis. The storage 210 may store EEG data obtained from EEG device 130 and enable the processor 220 to access the EEG data. In aspects, the storage 210 may be volatile memory and requires power to maintain stored information. In aspects, the storage 210 may be non-volatile memory and retains stored information when the computing device 200 is not powered. In aspects, the non-volatile memory includes flash memory. In aspects, the non-volatile memory includes dynamic random-access memory (DRAM). In aspects, the non-volatile memory includes ferroelectric random-access memory (FRAM). In aspects, the non-volatile memory includes phase-change random access memory (PRAM). In aspects, the storage 210 includes, by way of non-limiting examples, CD-ROMs, DVDs, flash memory devices, magnetic disk drives, magnetic tapes drives, optical disk drives, and cloud computing-based storage. In aspects, the storage 210 may be a combination of devices such as those disclosed herein.

The computing device 200 further includes a processor 220, an extension 230, a display 240, an input device 250, and a network card 260. The processor 220 executes instructions that implement tasks or functions of programs. When a user executes a program, the processor 220 reads the program stored in the storage 210, loads the program on the RAM, and executes instructions prescribed by the program.

The processor 220 may be a microprocessor, central processing unit (CPU), application specific integrated circuit (ASIC), arithmetic coprocessor, graphic processor, or image processor, each of which is electronic circuitry within a computer that carries out instructions of a computer program by performing the basic arithmetic, logical, control and input/output (I/O) operations specified by the instructions.

In aspects, the extension 230 may include several ports, such as one or more universal serial buses (USBs), IEEE 1394 ports, parallel ports, and/or expansion slots such as peripheral component interconnect (PCI) and PCI express (PCIe). The extension 230 is not limited to the list but may include other slots or ports that can be used for appropriate purposes. The extension 230 may be used to install hardware or add additional functionalities to a computer that may facilitate the purposes of the computer. For example, a USB port can be used for adding additional storage to the computer and/or an IEEE 1394 may be used for receiving moving/still image data. The extension 230, may enable the EEG device 130 to be connected to the computing device 200 via appropriate cables or via wireless communication devices.

In aspects, the display 240 may be a cathode ray tube (CRT), a liquid crystal display (LCD), or light-emitting diode (LED). In aspects, the display 240 may be a thin film transistor liquid crystal display (TFT-LCD). In aspects, the display 240 may be an organic light-emitting diode (OLED) display. In various aspects, the OLED display is a passive-matrix OLED (PMOLED) or active-matrix OLED (AMOLED) display. In aspects, the display 240 may be a plasma display. In aspects, the display 240 may be a video projector. In aspects, the display may be interactive (e.g., having a touch screen or a sensor such as a camera, a 3D sensor, a LiDAR, a radar, etc.) that can detect user interactions/gestures/responses and the like. In aspects, the display 240 is a combination of devices such as those disclosed herein.

A user may input and/or modify data via the input device 250 that may include a keyboard, a mouse, or any other device with which the user may input data. The display 240 displays data on a screen of the display 240. The display 240 may be a touch screen so that the display 240 can be used as the input device 250. In aspects, input device 250 may be the EEG device 130 for inputting EEG data into the computing device 200.

The network card 260 is used to communicate with other computing devices, wirelessly or via a wired connection. Through the network card 260, any communications can be made between the server 110 and the EEG device, or among any computing devices.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, C#, Delphi, Fortran, Java, JavaScript, machine code, operating system command languages, Pascal, Perl, PL1, python, scripting languages, Visual Basic, meta-languages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Certain aspects of the present disclosure may include some, all, or none of the above advantages and/or one or more other advantages readily apparent to those skilled in the art from the figures, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an aspect,” “in aspects,” “in various aspects,” “in some aspects,” or “in other aspects” may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for detecting neurological disorders based on an electroencephalogram (EEG) signal, the system comprising:
at least one processor; and
at least one memory storing instructions thereon which, when executed by the at least one processor, cause the system to:
access the EEG signal;
process the EEG signal to filter for a brainwave frequency band;
detect, via an EEG analytic engine, a brainwave pattern from the processed EEG signal based on the filtered brainwave frequency band; and
generate, via a synchronization pattern visual generator, a two-dimensional network pattern image of the detected brainwave pattern.

2. The system of claim 1, wherein the at least one memory stores further instructions which, when executed by the at least one processor, cause the system to generate a three-dimensional spatial graphic with the detected brainwave pattern superimposed thereon.

3. The system of claim 2, wherein the three-dimensional spatial graphic is a three-dimensional model of a brain having an amount of nodes equal to an amount of EEG sensors used to detect the EEG signal.

4. The system of claim 1, wherein the at least one memory stores further instructions which, when executed by the at least one processor, cause the system to display the two-dimensional spatial graphic.

5. The system of claim 1, wherein processing the EEG signal includes applying a Fourier transform.

6. The system of claim 1, wherein the at least one memory stores further instructions which, when executed by the at least one processor, cause the system to curate and transform time series data of the EEG signal.

7. The system of claim 6, wherein the at least one memory stores further instructions which, when executed by the at least one processor, cause the system to:
extract a non-linear invariant measure from the EEG signal;
generate a recurrence plot of the EEG signal; and
generate a correlation matrix relating the EEG signal to a plurality of sensors of an EEG device.

8. The system of claim 1, wherein a two-dimensional network pattern image of the detected brainwave pattern includes a map of a plurality of sensors, each sensor of the plurality of sensors configured to provide a portion of the EEG signal.

9. The system of claim 1, wherein the instructions to process the EEG signal include to generate a correlation matrix relating the EEG signal to a plurality of sensors of an EEG device.

10. The system of claim 9, wherein the EEG analytic engine detects the brainwave pattern based on the correlation matrix.

11. The system of claim 1, wherein the EEG analytic engine is a classical machine learning classifier, a convolutional neural network, a deep learning network, an associative, a non-associative, or a clustering machine learning system.

12. The system of claim 2, wherein the EEG signal is accessed in real-time from an EEG device; and the instructions further include at least one of:
generating a plurality of two-dimensional network pattern images of the detected brainwave pattern as a function of time; or
generating a plurality of three-dimensional spatial graphics with the detected brainwave pattern superimposed thereon as a function of time.

13. The system of claim 12, wherein the instructions to process the EEG signal include:
curate and transform time series data of the EEG signal;
extract at least one non-linear invariant measure from the EEG signal;
generate at least one recurrence plot of the EEG signal; and
generate at least one correlation matrix relating the EEG signal to a plurality of sensors of an EEG device.

14. A computer-implemented method for detecting neurological disorders based on an electroencephalogram (EEG) signal, the method comprising:
accessing an EEG signal;
processing the EEG signal to filter for a brainwave frequency band;
detecting, via an EEG analytic engine, a brainwave pattern from the processed EEG signal based on the filtered brainwave frequency band; and
generating, via a synchronization pattern visual generator, a two-dimensional network pattern image of the detected brainwave pattern.

15. The computer-implemented method of claim 14, further comprising generating a three-dimensional spatial graphic with the detected brainwave pattern superimposed thereon.

16. The computer-implemented method of claim 15, wherein the three-dimensional spatial graphic is a three-dimensional model of a brain having a plurality of nodes.

17. The computer-implemented method of claim 14, further comprising:
extracting a non-linear invariant measure from the EEG signal;
generating a recurrence plot of the EEG signal; and
generating a correlation matrix relating the EEG signal to a plurality of sensors of an EEG device.

18. The computer-implemented method of claim 17, wherein processing the EEG signal includes applying a Fourier transform.

19. The computer-implemented method of claim 14, designating, via the EEG analytic engine, a spatial correlation between a plurality of neural circuits associated with a plurality of sensors of an EEG device from which the EEG signal was sensed.

20. The computer-implemented method of claim 14, wherein the EEG signal is accessed in real-time from an EEG device; and
generating the two-dimensional network pattern image includes generating a plurality of two-dimensional network pattern images as a function of time.
